(19) United States
(12) Patent Application Publication
(10) Pub. No.: US 2008/0021361 A1
(43) Pub. Date: Jan. 24, 2008

(54) Anatomically configured design for pressure support, comfort, protection of a joint and or body part

(76) Inventor: Joseph O. Smith, Wellington, FL (US)

Correspondence Address: Dr. Joe Smith, 1921 South Club Drive, Wellington, FL, 33414, US

(21) Appl. No.: 11/489,048

(22) Filed: Jul. 19, 2006

(51) Int. Cl. A61F 13/06

(52) U.S. Cl. 602/61

(57) ABSTRACT

Our geometric devices “best mode” is made of an elastic-like material which can offer multi-directional support as opposed to the majority of the competition in which the support is limited to the material used rather than the design or integration of design and material. These competitive products systematically offer no more support and protection in the area of focus than in the area of non-focus, unless they have various materials, elastic or metal stays, holes and/or straps. Our revolutionary membrane of three, four or more overlapping circular like bodies (in part or whole) are integrated into our products to offer a multi-directional, 360° quadrant support, which may enhance specific support at the medial, lateral, inferior and/or superior part of the joint or body fitted part to assist the individual in reaching his or her optimal performance while offering protection, support, comfort and performance. The geometric design can be made of a material, which may be waterproof, act as shock absorber and provide multi-directional support creating a tremendous improvement in both support and performance. The geometric design (Impact Dome Technology 360°) can have one or more modified layers of overlapping circular like bodies (in part or in whole) providing a three dimensional fit.

### BACKGROUND OF THE INVENTION

1. Field of the Invention

This invention relates to an article of manufacture. More specifically, this invention is directed to a device and method for providing ergonomic and/or orthopedic support, comfort, balance, protection to a joint and/or body part or product. This device utilized in different products and applications may also provide support, comfort, protection, balance and/or adhesion to a product held and/or worn. This device can be utilized in different products and applications that may also provide support, comfort, balance, protection, balance and/or pressure integrated into a therapeutic device for treating the back and/or other parts of the body. This device utilized in different products and applications may also provide support, comfort, balance, protection integrated into a grip like handle that may minimize slippage, e.g. back, Full body brace, wrist, ankle, elbow, postural support, knee, uniforms, pants, shorts, helmets, shoes, seating, design gloves, footwear, adhesive bandages, pillows, tools, sports equipment and medical equipment. In the preferred embodiments of this invention, the device comprises an anatomically configured design having concentrated pressure support for a joint and/or body part.

2. Description of the Prior Art

The human body may be injured by strains, contusions, bruises, degenerative joint diseases, arthritis, and arthroscopic operations. Such injuries may cause hematomas and inflamed areas that need to be treated. In such cases, it has been found advantageous to apply a compress of some type to the injured area to effect more rapid healing, or prevent any further inflammation or injury. Typically, support for the body can be provided with an Ace bandage wrap, or an anatomically configured braces and supports which offer no more support in the area of focus than the areas of non-focus unless they have a stay, strap and/or hole. When protecting the body, back braces, full body braces, wrist braces, ankle braces, elbow braces, postural supports, knee braces, uniforms, apparel (pants, shorts, bras, etc.) helmets, shoes, seating design, gloves, footwear, adhesive bandages, pillows, tools, sports equipment and medical equipment have been made and provided with little attention and/or specifications as to design but rather material. The Impact Dome Technology 360°, an anatomically configured design that offers a superior improvement integrating both elastic-like material and design to create a better product in numerous applications that provide more support, comfort, protection, balance and in bandages offers better adhesive properties aiding in wound healing. The products that utilize this geometric design can help to absorb impact and deflect force to minimize injuries to the user.

A number of patents have been disclosed in the patent literature which discloses various devices for support or treatment of a weakened and/or injured joint.

### OBJECTS OF THE INVENTION

It is the object of this invention to remedy the above as well as related deficiencies in the prior art.

More specifically, it is the principal object of the present invention to provide an anatomically configured device for pressure support of a joint and/or body part, by integrating this device into a back brace, Full body brace, wrist brace, ankle brace, elbow brace, postural support, knee brace, apparel (uniforms, pants, shorts), helmets, shoes, seating design, gloves, footwear, adhesive bandages (wound care), pillows, tools, sports equipment and medical equipment. The “best mode” would be an elastic-like material similar to the initial patent filed.

It is another object of this invention to provide an anatomically configured device for pressure support of a joint and/or body fitted part, which may provide support inferior, superior, medial and lateral.

It is yet another object of this invention to provide an anatomically configured device or membrane of material within the back brace, full body brace, wrist brace, ankle brace, elbow brace, postural support, knee brace, apparel (uniforms, pants, shorts), helmets, shoes, seating design, gloves, footwear, adhesive bandages (wound care), pillows, tools, sports equipment and medical equipment for concentrated pressure support of a joint and/or body part with a multi-directional 360° quadrant support.

### SUMMARY OF THE INVENTION

The above and related objects are achieved by providing a membrane made of overlapping circular like bodies (in part or in whole) that can be providing a multi-directional support and may give an enhanced 360° quadrant support over the joint and/or body part or in relation to it. The “best mode” would be made of an elastic-like material as in the previous patent filing. The device of this invention can include an anatomically configured design, comprised of three, four or more overlapping circular-like bodies (in part or whole). These circular like bodies may provide a pressure concentrating means corresponding to one or more areas of (said) joint and/or body part providing a multi-directional support and a 360° quadrant support (hereafter also “pressure points”), so that it may focus or concentrate pressure support at the inferior, superior, medial and lateral points (said pressure). The device, thus, may provide for both ease of attachment to a product and increased or focused pressure, in a 360° quadrant support, inferior, superior, medial and lateral to the joint and/or body fitted part.

In the preferred embodiments of this invention, the pressure concentrating means of the composite comprises a delimited area having a plurality of rings of materials having relatively greater support than the product and/or apparel with which they are combined. For example, in the case of a helmet for support of the head, the areas with the pressure concentrating means can be located in several areas acting individually and/or jointly and may act as shock absorbing units that can reduce impact and deflect force upon impact. In the seating system the design may absorb impact as to minimize the potential insult to both the pelvis and the spine. In an adhesive bandage the (Impact Dome Technology 360°) multi-directional support can now offer an opportunity for better comfort, support, protection, balance and adhesion with this design. This design also offers the ability to dispense medication and/or other material to provide better solutions for the user. The overlapping circular like bodies (in part or whole) can offer significantly better support, protection, balance and adhesion when compared with other geometric designs. In a grip of a handle of a tool, medical, sports equipment, weaponry, etc. the (Impact Dome Technology 360°) multi-directional support now offers an opportunity for better comfort, support, protection, balance and/or control with this design. In a lumbar pillow the (Impact Dome Technology 360°) multi-directional support may now offer an opportunity for better comfort, support, protection, balance and/or control with this design when used for the low back or neck. In the lumbar pillow the design may offer spinal support in addition to medial and lateral lumbar support of the musculature.

### BRIEF DESCRIPTION OF THE DRAWING

FIG. 1, FIG. 2 and FIG. 3, are perspective views of a helmet with the support device of this invention.

FIG. 4 have perspective views of a seating design in a support device of this invention.

FIG. 5 is a perspective view of an adhesive bandage in a support device of this invention.

FIG. 6 is a perspective view of a tool with a support device of this invention.

FIG. 7 is a perspective view of a lumbar pillow design with the support device of this invention.

### DETAILED DESCRIPTION OF THE INVENTION INCLUDING PREFERRED EMBODIMENTS

The Figures which accompany this application, and referenced herein, depict representative embodiments of this invention. In each instance, the composite may comprise a layer of material with three, four or more overlapping circular-like bodies (in part or in whole) providing a multi-directional, 360° quadrant support having concentrated pressure support means arranged at pre-designated locations for pressure support in a back brace, full body brace, wrist brace, ankle brace, elbow brace, postural support, knee brace, apparel (uniforms, pants, shorts), helmets, shoes, seating design, gloves, footwear, adhesive bandages(wound care), pillows, tools, sports equipment and medical equipment. The layer of material can be anatomically configured onto a particular product, or, at a particular location providing a multi-directional, 360° quadrant support within the said product.

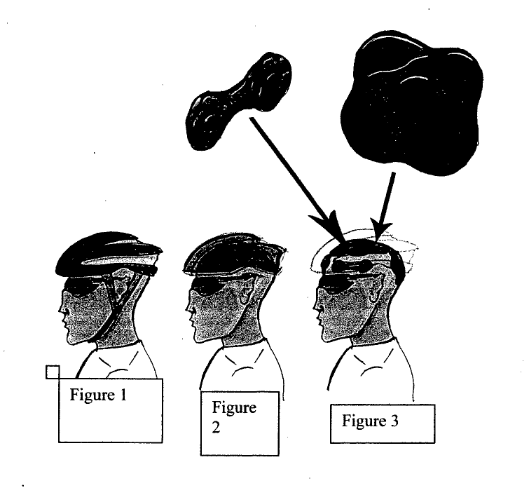

In FIG. 1, FIG. 2 and FIG. 3 the device of this invention is preconfigured in an anatomically shape suitable as a helmet support. Here the Impact Dome Technology can be utilized as shock absorbing units individually and/or jointly. These units can be positioned in headgear to minimize impact to the head resulting in decreased insult to both the head as well as the brain. FIG. 1 shows the outside of the helmet. FIG. 2 shows a cross-section of the helmet. FIG. 3 shows the inside of the helmet and the placement of the inserts (Impact Dome Technology).

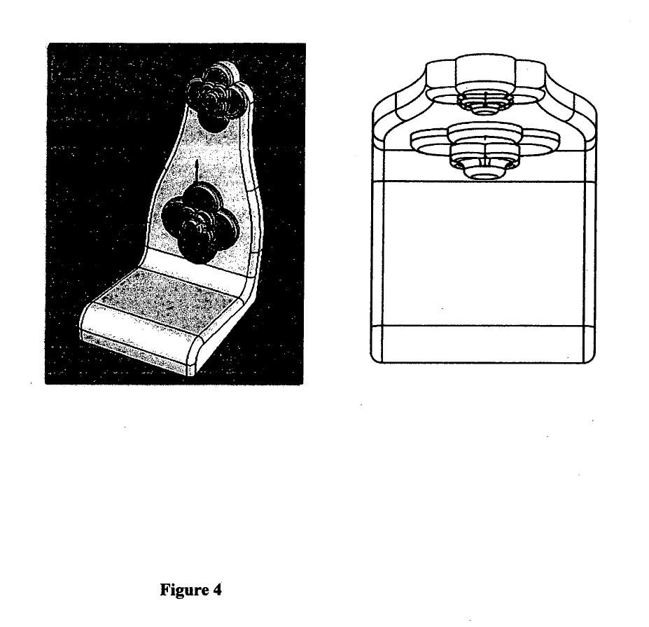

In FIG. 4, the device is preconfigured in an anatomical shape suitable a seating design support. The anterior view shows the placement of the inserts (Impact Dome Technology). The superior view shows the placement of the inserts (Impact Dome Technology).

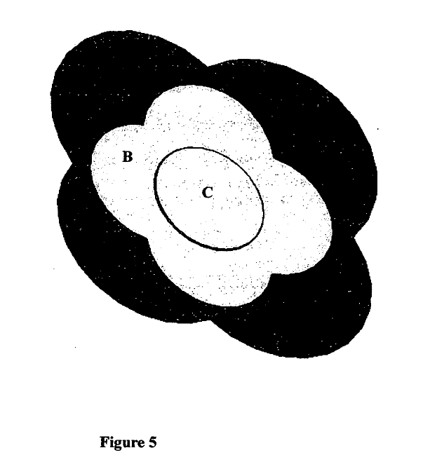

The FIG. 5 illustrates the adaptation of the concepts of this invention to a device (Impact Dome Technology) that is anatomically configured suitable for support of an adhesive bandage. Section A is the adhesive backed body of the bandage that can form the first layer. Section B can be the gauze the can form the second layer. Section C can be the medication in a compartment that forms the third layer.

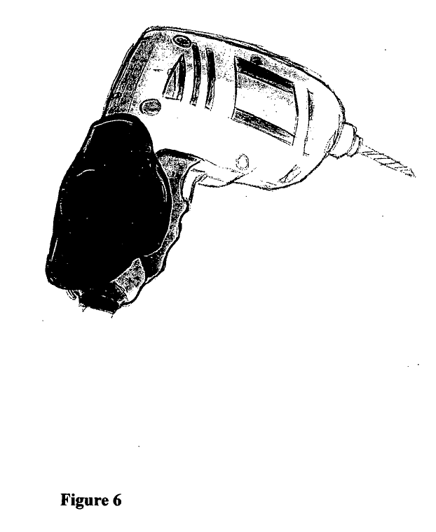

FIG. 6 illustrates the concept of this invention in the form of a grip on a power tool offering a greater multi-directional support. It shows the placement of the insert (Impact Dome Technology) that can minimize slippage on the handle and help to absorb vibration and shock.

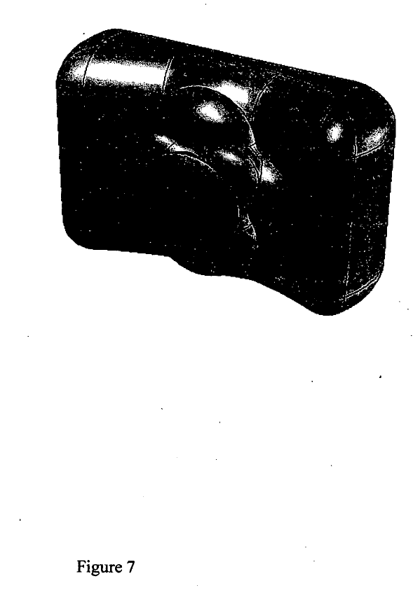

FIG. 7 illustrates the concept of this invention in a lumbar support pillow that offers spinal support as we as medial and lateral muscle support. The view shows an anterior positioning.

What is claimed is:

1. In the device for pressure support wherein the (said) device comprises a material that is or can be anatomically configured to a joint and/or body part, the improvement comprising:
a device comprising a composite of material of the membrane of three, four or more overlapping circular like bodies (in part or in whole) within or on the product and at least one pressure concentrating means located at a pre-designated areas on the (said) device, lends additional enhanced support or focused pressure to at one or more locations on the device where the (said) pressure concentrating means are located.

2. The improved ensemble of claim 1, wherein the (said) device is anatomically configured to provide support that may minimize impact, deflect force and/or provide more support, comfort, protection, control, balance and/or adhesion for a helmet, bandages, (tools, sports/medical equipment, weaponry), lumbar pillow and/or cervical/lumbar/seating pillow.

3. The improved ensemble of claim 1, wherein the said) device is anatomically configured to provide increased focal support in a helmet, bandages, (tools, sports/medical equipment, weaponry), apparel, lumbar pillow and/or cervical/lumbar/seating pillow.

4. The improved ensemble of claim 1, wherein the (said) device is in the form of the membrane (a design) which may have pressure concentrating means periodically spaced at pre-determined locations.

5. The improved ensemble of claim 1, wherein the (said) pressure concentrating means of the (said) device or membrane comprise a plurality of overlapping circular like bodies (in part or in whole) that may provide enhanced 360° quadrant support on the location, joint, and/or body part where design (membrane) is placed.
